(19) United States
(12) Patent Application Publication
(10) Pub. No.: US 2019/0045720 A1
(43) Pub. Date: Feb. 14, 2019

(54) Plant Growth Acceleration System and Methods

(71) Applicant: CO2Gro, Inc., Toronto (CA)

(72) Inventors: John Archibald, Grimsby (CA); Sam Kanes, Toronto (CA)

(21) Appl. No.: 16/101,377

(22) Filed: Aug. 10, 2018

Related U.S. Application Data
(60) Provisional application No. 62/544,541, filed on Aug. 11, 2017.

(51) Int. Cl. A01G 7/02; B05B 7/32; A01H 6/14; A01H 6/28

(52) U.S. Cl. CPC A01G 7/02; A01H 6/28; A01H 6/1472; B05B 7/32

(57) ABSTRACT

The present invention provides novel and effective compositions and methods for promoting the growth of green photosynthetic plants, particularly higher plants. The method relies on applying compounds comprising carbon dioxide infused water as a foliar spray to the plant and its leaves, where the compound increases intracellular carbon dioxide levels in an amount sufficient to inhibit photorespiration within the plant cells and thus enhance plant growth.

### BACKGROUND

The present invention relates generally to methods and compositions for stimulating and maintaining enhanced growth in plants. More particularly, the present invention relates to plant growth formulations which contain carbon dioxide infused water plus optionally additional nutrients, which compositions are able to enhance carbon fixation and growth in plants.

Photosynthesis is the process by which photosynthetic plants utilize solar energy to build carbohydrates and other organic molecules from carbon dioxide and water. The conversion of carbon dioxide to such organic molecules is generally referred to as carbon fixation and.

In U.S. Pat. No. 6,209,855 the invention described involved the concept of gas infusion. The hydrophobic nature of a hydrophobic microporous hollow fibre membrane established a stable interface between an aqueous phase on one side of the fibre and a gas phase on the other. The interface remains stable so long as there does not exist a pressure differential between the phases in excess of the ‘breakthrough’ pressure required to ‘push’ the aqueous phase through the micropores, or the gas pressure exceeds the liquid pressure to such an extent as to bubble into the liquid phase. This stable interface can be used to transfer carbon dioxide mass (CO2, or CO₂) from one phase to the other. The disclosure of this patent is incorporated herein by reference.

CO₂ gas-infused water has previously been used to increase the growth rates of algae and to treat human ailments. In one embodiment, previous work examined how the human foot absorbs oxygen (O2, or O₂) when immersed in water that has a high dissolved O₂ content. Compared with the tap water condition, tissue oxygenation index was raised by 3.5%±1.3% higher in feet treated for 30 min with O2-infused water. This effect persisted after treatment, as skin PO2 was higher in feet treated with O2-infused water at 2 min (237±9 vs. 112±5 mm HG) and 15 min (131±1 vs. 87±4 mm HG) post-treatment. When blood flow to the foot was occluded for 5 min, feet resting in O2-infused water maintained a 3-fold higher O2 consumption rate than feet treated with tap water (9.1±1.4 vs. 3.0±1.0 μL·100 g−1·min−1). Thus, skin was found to absorb appreciable amounts of O2 from O2-infused water. Can. J. Physiol. Pharmacol. 90: 1-10 (2012).

U.S. Pat. Nos. 6,436,290 and 7,537,200 claim an apparatus for controlling the dissolved gas content of an aqueous liquid containing dissolved gas, comprising a microporous hydrophobic hollow fibre membrane useful according to the present invention and are specifically incorporated by reference herein in its entirety.

U.S. Pat. No. 5,597,400 describes the use of foliar spay applications of methanol to increase plant growth.

U.S. Pat. No. 5,487,835, describes methods of mixing CO₂ gas and water at various pressures to achieve changes in the pH level.

### SUMMARY OF THE PRESENT INVENTION

Fertilizers for higher plants generally include nitrogen, phosphorus, and potassium, which are referred to as primary nutrients or macronutrients. Fertilizers often further include certain secondary nutrients, such as iron, sulfur, calcium, and magnesium, as well as various minerals and micronutrients. Heretofore, little attention has been paid to providing formulations which act directly to enhance carbon fixation in higher plants. Conventional fertilizer formulations have generally been directed at the delivery of the recognized primary, secondary, and micronutrients, but have usually not included a carbon source and, in particular, have not included a carbon source intended to enhance carbon fixation.

In traditional methods of outdoor gassing, it is estimated that over half of CO₂ gas is usually lost to the air, potentially contributing to the ‘greenhouse effect’ and global warming. Indoor gassing is typically done at levels that are not ideal for worker health and safety, while also losing significant amounts of CO₂ to ventilation

It would be desirable to provide improved methods, compositions and formulations for promoting plant growth by enhancing the rate of carbon fixation within the plant. It would be desirable for such methods, compositions and formulations to be relatively convenient, safe and simple to apply. It would be particularly desirable for such methods, compositions and formulations to be effective with most or all higher leafy plants. Additionally, it would be desirable for such methods, compositions and formulations to promote rapid growth and maturing of the treated plant.

It would be desirable to provide improved methods, apparatuses, compositions and formulation for promoting plant growth by enhancing the leaf conductance of gases, such as carbon dioxide, in plants, and in particular, the cuticular conductance, the stomatal conductance, or both. It would be desirable if such methods, apparatuses, compositions and formulations could be used with minimal loss of CO₂ gas into the atmosphere.

The present invention addresses, in whole or in part, each of the above desirable objectives. The present invention further provides convenient compositions and formulations, as well as methods for applying said compositions and formulations, such as applying the compositions and formulations as a foliar spray.

### DETAILS OF THE PRESENT INVENTION

The present invention provides novel and effective compositions, formulations and methods for promoting the growth of green photosynthetic plants, particularly higher plants. The method relies on applying compounds comprising carbon dioxide infused water as a foliar spray to the plant and its leaves, where the compound increases intracellular carbon dioxide levels in an amount sufficient to inhibit photorespiration within the plant cells and thus enhance plant growth.

The methods, compositions and formulations of the present invention are effective with virtually all photosynthetic plant species having leaves or other surfaces capable of receiving foliar sprays, particularly higher plants. “Higher” plants include all plant species having true stems, roots, and leaves, thus excluding lower plants, e.g. yeasts, algae and molds.

Suitable plants which may benefit from applications according to the present invention include crop plants, such as rice, peanuts, barley, broccoli, cauliflower, celery, mint, grapes, potato, eggplant, zucchini, squash, cucumber, bean, lettuce, collard greens, chard, sugar beet, carrot, radish, onion, leek, kale, tobacco, cannabis, alfalfa, flaxseed, oats, soybean, turnip, parsnip, capsicum, pepper, tomato, cabbage, lettuce, spinach, parsley, and the like; melons, gourds, squash, pumpkin and the like; herbs and berries such as coffee, tea, allspice, anise, basil, bay laurel, blackberry, blueberry, borage, caraway, cardamom, catnip, chives, cilantro, chervil, chicory, cinnamon, clove, clover, comfrey, coriander, cumin, dill, elderflower, fennel, fenugreek, garlic, ginger, ginseng, hawthorn, horseradish, jasmine, juniper, lemon grass, lavender, lemon verbena, licorice, lovage, lemon balm, mace, marjoram, milk thistle, mint, mustard, nutmeg, oregano, paprika, pepper, poppy seed, raspberry, rosemary, saffron, sage, salad burnet, savory, geranium, sorrel, star anise, stevia, St. John's wort, strawberry, sumac, tabasco, tarragon, thyme, turmeric, valerian, vanilla, wasabi, watercress, wintergreen, yerba buena, wheat, hemp, rape, corn and the like; flowering plants, such as rose, coleus, chrysanthemum, ginkgo bilboa, poppy, African violets, bougainvillea, oleander, eucalyptus, hibiscus, gardenia, jasmine, camellia, marigold, daisy, stock, vinca, gerbera, carnation, cyclamen, peony, shooting star, bird-of-paradise, forget-me-not, and the like; fruit and berry trees, such as apple, avocado, banana, coconut, mango, olive, orange, pear, plum, peach, cherry, citrus, and the like; and forest trees, such as pine, holly, chestnut, beech, redwood, cypress, juniper, elm, birch, palm, tea tree, and the like. Suitable plants also include germinated, partially germinated, sprouted or partially sprouted microgreens, seeds, roots and sprouts. The above list is intended to be exemplary and not intended to be exclusive.

The present invention is particularly well suited for use in arid and semi-arid climates, where irrigation is the primary method for delivering water to plants. The methods of the present invention can also be used in concert with any irrigation system, and can be adapted for use in greenhouses and other growing environments. The present invention provides methods by which CO₂ gas is dissolved in water, and the CO₂-infused water is then applied onto the leaves of plants. Because of the high concentration of CO2 created in the microenvironment at the surface of the leaf, CO₂ gas is quickly absorbed into the leaves, and the vast majority of the CO₂ is absorbed by the plants, with little loss of CO₂ into the atmosphere.

The present invention provides substantial benefits in increasing the growth of plants, especially leafy vegetables and flowers. With increased growth, a shorter growing season, or shorter time to harvest, may be required. The present invention provides multiple additional advantages including increased control of pathogens, mold, slime and algae, as well as providing a degree of protection against insects and pests, and greatly reducing or preventing spoilage and crop loss due to wilting, desiccation and dry rot. Each of these advantages provides the opportunity for significant cost savings, increased productivity, and improved versatility in land use.

The methods, compositions and formulations of the present invention may be used to promote growth in tissues of either juvenile or mature plants. Generally, however, it is desirable that the plants include at least two true leaves beyond the cotyledon or cotyledon pair (i.e. the “seed leaves”). Improved growth occurs as a result of several pathways for the metabolism of CO₂-infused water which benefit from reduced photorespiration. In addition to such enhanced growth, treatment of plants with the compositions of the present invention may result in an enhanced turgidity.

When employed in greenhouses and in artificially lit growing areas, the methods of the present invention may further result in increased factory efficiency, lessening the number of kilowatt-hours per plan consumed by expensive lighting, hence significantly reducing utility costs. Additionally, the present invention allows the replacement of gas containment and pumping systems with a liquid spray which folds CO₂ supplementation into irrigation, lowering equipment and maintenance costs.

Importantly, the methods of the present invention enhance the ability to grow plants, flowers and trees under organic conditions. That is, using sustainable methods, such as use of cover crops, biodiversity, crop rotation and renewable resources for the fertilization of soil and plants, while minimizing the use of external and off-farm inputs, without the use of synthetic pesticides, fertilizers and other materials, such as hormones and antibiotics.

According to the invention, we have found that, designed, built and operated correctly, gas infusion can be used to increase dissolved carbon dioxide content of an aqueous liquid to previously unachieved levels, while simultaneously lowering the total dissolved gas pressure (TG) of the aqueous liquid, and do it all economically. We have called this process, “controlled atmosphere gas infusion.” Water highly infused with CO₂ can be used to dramatically increase the growth rate of certain leafy plants, such as those disclosed above and specifically including lettuce, tobacco and Cannabis sativa or indica.

An important principle that is not disclosed in prior patents, or in the scientific literature, is the concept and utility of using gas infusion to create highly CO₂ infused water which is fed to plants via foliar feeding to increase the growth of economically important leafy plants.

In conditions of relatively abundant nutrients, sun and water, plants primarily absorb and lose water and gases, such as CO₂, through their stomata. Under such conditions, cuticular conductance of CO₂ is a relatively small fraction compared to the cuticular conductance of water vapor, which is smaller than CO₂. The net result is that the diffusion path for CO₂ is strongly stomatal, while the path for water vapor involves both the stomata and the cuticle. However, as leaves become darkened or dehydrated, their stomatal apertures begin to close, such that water loss and exchange of CO₂ becomes increasingly dependent upon the cuticle. Boyer et al. (1997) Plant Physiol. 114:185-191.

Foliar feeding is a method of feeding plants by applying liquid fertilizer directly to their leaves rather than through their roots. Plants are able to absorb essential elements through their leaves. The absorption takes place through their stomata and also through their epidermis. Transport is usually faster through the stomata, but total absorption may be as great through the epidermis.

Foliar feeding was earlier thought to damage tomatoes, but has now become standard practice. Addition of a spray enhancer can help nutrients stick to the leaf and then penetrate the leaves.

Foliar application has been shown to avoid the problem of leaching-out in soils and prompts a quick reaction in the plant. Foliar application of phosphorus, zinc and iron brings the greatest benefit in comparison with addition to soil where phosphorus becomes fixed in a form inaccessible to the plant and where zinc and iron are less available. See https://en.wikipedia.org/wiki/Foliar feeding (accessed Jul. 31, 2017).

Foliar feeding has been used as a means of supplying supplemental doses of minor and major nutrients, plant hormones, stimulants, and other beneficial substances. Observed effects of foliar fertilization have included yield increases, resistance to diseases and insect pests, improved drought tolerance, and enhanced crop quality. Plant response is dependent on species, fertilizer form, concentration, and frequency of application, as well as the stage of plant growth. Foliar applications may be timed to coincide with specific vegetative or fruiting stages of growth, and the fertilizer formula is adjusted accordingly for best results. In terms of nutrient absorption, foliar fertilization can be from 8 to 20 times as efficient as ground application. See Foliar Fertilization George Kuepper, NCAT Agriculture Specialist, Published 2003, ATTRA Publication #CT135 accessed at https://attra.ncatorg/attra-pub/summaries/summary.php? pub=286#intro Jul. 31, 2017.

According to the present invention, foliar feeding may be used to provide CO₂ infused water to economically important plants and dramatically and surprisingly increase the growth rates of the plants.

Foliar provision of CO₂ according to the present invention may also be accompanied by the addition of sufficient nutrients to account for the increased growth rates achieved by the addition of infused CO₂. According to one aspect of the invention, water is infused with CO₂ at pressure, temperature and other conditions sufficient to achieve high levels of CO₂ infusion in the water. According to another aspect of the invention, water is infused with CO₂ at pressure and other conditions sufficient to achieve saturation levels of CO₂ infusion in the water. According to another aspect of the invention, water is infused with CO₂ at pressure and other conditions sufficient to achieve supersaturation levels of CO₂ infusion in the water.

While not being bound by any particular theory, the inventors theorized that growth of plants could be enhanced utilizing the methods of this invention in which water highly infused with carbon dioxide is used in combination with methods, compositions and apparatuses for foliar misting and feeding, which would enhance leaf conductance of gases, and, in particular, of conductance of CO₂, through both the stomata and the cuticle. The mechanism for doing so is believed to lie in altering the ratio of CO₂ gas in water vaper in a local environment on the leaf. By infusing water with CO₂ prior to foliar spraying, the present invention produced surprising and unexpected results, significantly increasing the uptake of CO₂ into the leaf over both atmospheric [approximately 250-350 mg/liter or ppm CO₂ in air] and supplemented atmospheric [for example, approximately 1000-2000 mg/l CO₂ in controlled greenhouses] conditions. While the rate of uptake is highest when occurring throughout the leaf via the entire epidermis, stomata and cuticle, the present inventors have surprisingly found that uptake of CO₂ is enhanced even when the stomata is blocked. Accordingly, using the methods of the present invention, the rate of CO₂ entering the leaf can be increased via both the stomata and across the cuticle through the epidermis.

Using the methods of the present invention, surprisingly, near instantaneous increases of CO₂ transfer can be measurably observed. For example, the methods of the invention result in increased CO₂ conductance, as measured through a porometer, which provides the most direct measurement of CO₂ uptake. Additionally, the methods of the present invention result in surprising increases in chlorophyll A, which are consistent with the plant's increased ability to process more CO₂ into carbohydrate, meeting the increased physiological needs for plant or leaf growth, because of its (CO₂'s) increased availability. The methods of the invention produce surprising and unexpected enhancements in vegetation and leaf biomass, consistent with the availability of increased carbohydrate reserves to the meristematic tissue of the plant. Each of the above results have been identified in short term and long term (to harvest) testing, and demonstrate that the present invention produces unexpectedly substantial, surprising and significant increases in CO₂ uptake, which can significantly enhance the growth and health of plants.

By coupling CO₂ infusion technology with foliar misting, the present invention produces a surprisingly rich microenvironment that is highly targeted to the leaf. This allows higher CO₂ concentrations locally to be experienced by the plants, which cannot otherwise be achieved atmospherically without potentially endangering animal and human health. The present invention further provides more efficient delivery of CO₂, as the water vapor infused with CO₂ is applied in a targeted fashion across the entire plant leaf surface area, rather than the entire atmosphere. Ultimately, the invention provides a method for highly efficient carbon sequestration in terrestrial environments.

There are additional technologies that are especially applicable to foliar fertilization: one is the use of electrostatic sprayers, which impart a charge to the spray particles and cause them to adhere more readily to plants, another is the use of spray enhancers. In certain embodiments, the foliar spray is optimized for control of water droplet size.

In one preferred embodiment of the present invention, foliar sprays are finely atomized. This can be managed by increasing sprayer pressure or by using a mist blower. Absorption is increased when sprays also reach and coat the undersides of leaves. This is where most of the plant's stomata are located. In another embodiment, air temperatures should be below 80° F., as absorption at higher temperatures may be reduced because plant stomata are closed. In certain embodiments absorption may be enhanced when growing conditions are humid and moist. The presence of heavy dew on the leaves may facilitate foliar feeding. As noted above, addition of a spray enhancer may be beneficial. For example, addition of a spray enhancer such as a surfactant to the solution decreases surface tension on the leaf and may increase absorption. Other spray enhancers may increase the wetting of the leaves, and thereby increase penetration of CO₂ into the plant.

According to the present invention, water is infused with CO₂ under conditions sufficient to result in CO₂ concentrations in water in excess of atmospheric concentration [typically expressed as 250-350 milligrams CO₂ per liter air (mg/1). Accordingly, in certain embodiments of the invention, water is infused with CO₂ under conditions sufficient to result in CO₂ concentrations of greater than about 0.37 mg CO₂/liter water; greater than about 0.4 mg CO₂/liter; greater than about 0.5 mg CO₂/liter; greater than about 0.6 mg CO₂/liter; greater than about 0.7 mg CO₂/liter; greater than about 0.8 mg CO₂/liter; greater than about 0.9 mg CO₂/liter; greater than about 1.0 mg CO₂/liter; greater than about 1.2 mg CO₂/liter; greater than about 1.5 mg CO₂/liter; greater than about 1.8 mg CO₂/liter; or greater than about 2.0 mg CO₂/liter (aq.). In certain embodiments, the concentration of CO₂ is controlled so as to fall within a desired range. Accordingly, in certain embodiments of the invention, water is infused with CO₂ under conditions sufficient to result in CO₂ concentrations falling within the range of about 0.37 mg/l to about 2400 mg/1; about 0.6 mg/l to about 2200 mg/1; about 0.7 mg/l to about 2000 mg/1; about 0.8 mg/l to about 2000 mg/1; or within the range of about 1.0 mg/l to about 2000 mg/l.

Both temperature and salinity affect the amounts of CO₂ that can be dissolved in water. In most increased plant growth circumstances, salinity will never be a factor. Temperature affects the amount of CO₂ water can retain. However, using the present invention, the ranges set out herein above are readily attained at typical growth temperatures.

The infused water is then sprayed or misted in a manner so as to cover the entire leaf or plant, or planted area. If desired, spraying or misting can be designed so that the CO₂ infused water or water vapor additionally covers the underside of the leaf, plant or planted area.

According to one aspect of the present invention water infused with CO₂ is sprayed in a manner that provides coverage of a plant, two or more plants, or a field of plants with sufficient CO₂, sufficient to enhance leaf conductance of CO₂, such that the ratio of CO₂ conductance:water vapor conductance exceeds 5.7%.

In another aspect of the present invention, methods are provided for water that is infused with CO₂ to achieve saturated or supersaturated levels.

According to one aspect of the invention, a method is provided for controlling the dissolved gas content of an aqueous liquid containing dissolved CO₂, comprising providing a microporous hydrophobic hollow fibre membrane, to provide at equilibrium a stable interface between an aqueous liquid phase containing dissolved CO₂ on one side of the membrane and a gaseous phase on the other side of the membrane and controlling the aqueous and gaseous phase pressure, such that the gaseous phase pressure is up to but not exceeding the aqueous phase pressure.

According to another aspect of the invention, an apparatus is provided for controlling the dissolved gas content of an aqueous liquid containing dissolved CO₂ gas, comprising a means for mixing gas with water at a desired concentration. One such means comprises microporous hydrophobic hollow fibre membrane, to provide at equilibrium a stable interface between an aqueous liquid phase containing dissolved CO₂ gas on a first side of the membrane and a gaseous phase on an opposite side of the membrane, means providing preferably substantially countercurrent aqueous liquid phase and gaseous phase flow paths on opposite sides of the membrane, means for supplying an aqueous liquid phase containing dissolved CO₂ gas to the first side of the membrane, means for controlling the flow feed rate of the aqueous liquid phase, means for controlling the aqueous liquid phase inlet pressure, means for supplying a gaseous phase to the other side of the membrane, means for controlling the gaseous phase inlet pressure, means for removing gaseous phase from the apparatus, and means for removing aqueous phase from the apparatus. Other means suitable for use in the present invention include CO₂ injection, and may include the use of porous stone air diffusers and venturi diffusers.

According to another aspect of the invention, the method or apparatus for controlling dissolved gas content of aqueous liquid and foliar spraying of such infused liquids may include controls for one or more additional parameters, such as injection pressure, air pressure, flow feed rate, temperature, pH, droplet size; duration and frequency of spraying;

According to yet another aspect of the invention, the mass transfer of the CO₂ gas from the gaseous phase into the liquid phase occurs by absorption.

One object of a process according to the present invention may be to increase the dissolved carbon dioxide content of water for use in hydroponics by using pure carbon dioxide. In certain embodiments of the present invention, therefore, it would be preferable for that process to utilize as much of the CO₂ as possible with little wastage.

### EXAMPLES

### Example 1

A series of experiments were performed which demonstrate the impact of CO₂ delivered in supersaturated water to stomata via foliar misting. The first of these experiments was initiated with seeding plants being prepared for foliar spray exposure or other control/null treatments. The initial experimentation was designed to identify the impacts of long term (germination to harvest) exposure CO₂ enriched foliar spray. It was decided to test if short term physiological modification in plants could be observed in response to CO₂ enriched foliar spray, while longer term experiments were underway.

In an initial trial, romaine lettuce (the target species for the first experiment) was misted with CO₂ enriched water every 15 minutes for a four-hour period. During each 15-minute interval, a 5 mm disc was cut from the lettuce leaf for chlorophyll A extraction. Each disc represented approximately 1 mg of plant material. Chlorophyll A was extracted using a 90% acetone solution and then quantified using standard methods with a Turner TD-700 Fluorimeter. Results of this experiment showed a 4-fold sustained increase in chlorophyll A in cuttings from pants treated with CO₂ enriched water in accordance with the present invention over control cuttings from the first to final 15 minute misting interval.

TABLE 1

Chlorophyll A (parts per billion)

| | Trial 1 | Trial 2 | Control 1 | Control 2 |
|---|---|---|---|---|
| 1 | 788.7 | 706.8 | 182.1 | 145.8 |
| 2 | 506.6 | 483.2 | 149.8 | 189.9 |
| 3 | 544.5 | 540.9 | 132.8 | 133.9 |
| 4 | 1100 | 1030 | 141.8 | 177.6 |
| 5 | 725.9 | 630.3 | 158.3 | 170.2 |
| 6 | 520 | 556.5 | 171.1 | 167.1 |
| 7 | 1001 | 949.6 | 177.3 | 145.1 |
| 8 | 671.2 | 406.4 | 199.4 | 141.4 |
| 9 | 703.8 | 847.6 | 137.6 | 187.4 |
| 10 | 849.7 | 492.3 | 168.0 | 146.4 |
| 11 | 360.6 | 390.8 | 168.5 | 171.3 |
| 12 | 1005 | 963.8 | 182.3 | 188 |

Leaves misted every 15 minutes for 3 hours. Two 5 mm circles cut from leaf. Chlorophyll A extracted
Note values may not be directly comparable due to variations in water content of sprayed and non

The experiment was repeated a second time. However, this experimental replicate was run at 15-minute intervals for 2 hours. Chlorophyll A was measured using an Apogee MC-100 Chlorophyll Concentration meter. This meter allowed chlorophyll A to be estimated without cutting the leaf or damaging the plant in any other fashion. Chlorophyll A is reported as a unit area rather than extraction by weight and the meter estimates chlorophyll A directly by contact on the leaf's surface. Results from this second experiment were consistent with the first. A statistically significant (p=0.010477, t-test) increase (˜30%) in chlorophyll A per m² of leaf surface area was observed in plants treated according to the present invention over the duration of the experiment beginning with the first 15 min interval.

TABLE 2

| | Plant A | Plant B |
|---|---|---|
| Minutes | (Sprayed | (Control) |
| 15 | 8 | 8 |
| 30 | 8.5 | 8.3 |
| 45 | 13.1 | 8.3 |
| 60 | 13.1 | 8 |
| 75 | 11 | 8.6 |
| 90 | 9.1 | 8.3 |
| 105 | 10.3 | 8.5 |
| 120 | 13.1 | 8 |

Taken every 15 minutes; pH 3.75
Leaves misted every 15 minutes dor 3 hours.
Chlorophyll per unit area (specifically μmol/m²)
Best estimate to compare control and sprayed

Notable in these initial experiments is the rapidity of physiological response seen in CO₂ exposed plants. This data is encouraging and consistent with the hypothesis of significant growth enhancement with CO₂ delivery via foliar spray.

A third experiment was conducted, utilizing an SC-1 Leaf Porometer (ICT International) to measure stomatal conductance. Stomatal conductance is an estimate of the rate of CO₂ entering and/or water vapor exiting leaf stomata. This metric is likely the most directly related to measuring increases of CO₂ availability to plants via super saturated water deposited near the stomata.

Three experiments were run with the porometer. In all experiments chlorophyll A concentration was measured (Apogee MC-100) with stomatal conductance (ICT International SC-1). In the first, both metrics were quantified every 20 minutes for 100 minutes. Two treatments were considered: 1) CO₂ enriched foliar spray and 2) no spray. Data for each metric was compared between treatments using a t-Test for equal means. Both chlorophyll A (p=0.0077) and stomatal conductance (p=0.0131) showed significant increases in the CO₂ exposed treatments.

The second and third experiment were identical in treatments and metrics quantified. The only difference was in duration of the experiment. This is a result of the time it takes to acquire a stomatal conductance estimate in comparison to chlorophyll A. In the second experiment the duration was 2 hours and 20 minutes; and the third lasted 4 hours. Treatments for these experiments included: 1) CO₂ enriched foliar spray, 2) unenriched foliar spray, and 3) no spray. The unenriched foliar spray treatment was added to test the hypothesis that water vapor alone could explain the results from prior experiments. In both experiments chlorophyll A was measured for 5 randomly selected leaves every 10 minutes immediately following treatments which were also applied every 10 minutes. Stomatal conductance was measured each hour for each treatment. Both experiments were consistent in showing higher chlorophyll A content and higher stomatal conductance in CO₂ exposed treatments. ANOVA was used to compare chlorophyll A data in both experiments and stomatal conductance in the third experiment (only two estimates existed for experiment 2 making statistical comparison impossible). Significant differences existed between CO₂ exposed treatments for chlorophyll A (p=0.00057, exp2 and p=0.0000005.5, exp3) and stomatal conductance (p=0.00000074). Notably, no significant difference existed between unenriched spray and no spray treatments, strongly suggesting that CO₂ availability was the factor increasing both chlorophyll A and stomatal conductance, rather than any effect of foliar application of water alone.

TABLE 3

| | Treatment | | |
|---|---|---|---|
| MIN | CO₂ Foliar Spray | Unenriched Foliar Spray | No Spray |
| 10 | 5.96 | 5.64 | 4.98 |
| 20 | 6.40 | 4.68 | 4.98 |
| 30 | 6.22 | 4.98 | 5.26 |
| 40 | 6.52 | 6.22 | 4.74 |
| 50 | 5.46 | 4.90 | 602 |
| 60 | 6.52 | 5.14 | 5.26 |
| 70 | 5.60 | 5.46 | 5.06 |
| 80 | 6.42 | 4.48 | 5 |
| 90 | 4.56 | 5.76 | 4.42 |
| 100 | 4.48 | 5.18 | 4.54 |
| 110 | 5.58 | 5.14 | 4.72 |
| 120 | 5.90 | 4.94 | 4.64 |
| 130 | 5.92 | 5.68 | 5.18 |
| 140 | 5.46 | 4.98 | 4.66 |

Chlorophyll A units=μmol/m2 leaf
Averages: CO₂ Enriched Foliar Spray: 5.785714286
   Unenriched Foliar Spray: 5.227142857
   No Spray: 4.961428571
ANOVA same means: p=0.00057
Tukey's Same Mean
Treatment 1 vs. Treatment 2: p=0.0196 (significant)
Treatment 1 vs. Treatment 3: p=0.00056 (significant)
Treatment 2 vs. Treatment 3: p=0.378 (not significant)
In this experiment treatments were applied every 10 minutes for 2 hours and 20
Chlorophyll A measurements were taken from 5 random leaves from plants under each treatment every 10 minutes.
Treatments included CO₂ enriched foliar spray, unenriched foliar spray, and no spray.

TABLE 4
Stomatal Conductance Data

| | CO₂ Enriched | Unenriched | |
|---|---|---|---|
| Minutes | Foliar Spray | Foliar Spray | No Spray |
| 70 | 915.7 | 91.9 | 85.6 |
| 140 | 1457 | 226.3 | 122.1 |
| Avg | 1186.35 | 159.1 | 103.85 |

Stomatal Conductance Units=μmol m−2 s−1 gas
In this experiment treatments were applied every 10 minutes for 2 hours and 20 minutes.
Stomatal Conductance measurements were taken from a random leaf from a plant under each treatment at 70 and 140 minutes.
Treatments included CO₂ enriched foliar spray, unenriched foliar spray, and no spray.

### Example 2

A field experiment was conducted on the effects of CO₂-enriched foliar spray on the growth of cannabis. Three distinct species of cannabis plants were tested: Indica, Sativa, and a hybrid species known as ‘Great White Shark.’ Two hundred forty plants of each species were included in the test, half treated and half control.

TABLE 5
Cannabis Field Growth Experiments

| | | | | | Hybrid | |
|---|---|---|---|---|---|---|
| | Indica | | Sativa | | ‘Great White Shark’ | |
| | CO₂ | | CO₂ | | CO₂ | |
| | sprayed | Control | sprayed | Control | sprayed | Control |
| Average | 148% | 100% | 140% | 100% | 151% | 100% |
| plant size | | | | | | |
| Average leaf | 197% | 100% | 208% | 100% | 188% | 100% |
| size | | | | | | |
| Vegetative | 76% | 100% | 75% | 100% | 78% | 100% |
| time | | | | | | |
| Total bud | 120% | 100% | 124% | 100% | 125% | 100% |
| weight | | | | | | |

Lights, nutrients and water amount and frequency were the same for all plants.
No CO2 foliar applications were made during budding

Additional analyses from cannabis commercial indoor growth trials with indica showed that the buds had significantly greater tetrahydrocannabinol (THC) content, as well as increases in other active agents including cannabidiol (CBD) and cannabinol (CBN). The above increases in plant, leaf and bud growth, and reduction in vegetative time, are estimated to increase the value of the crop by an estimated 45-60%.

### Example 3

The above described results demonstrate rapid increases in chlorophyll A and stomatal conductance in response to exposure to foliar spray with CO₂-infused water. Values for stomatal conductance were an order of magnitude greater in foliar sprayed leaves in comparison to control leaves. These results suggested that CO₂ may be entering the leaf independent of the stomata.

In order to test the hypothesis, further experiments were conducted. In these experiments, the leaf's upper surface was treated instead of the bottom side. Stomata are typically scarce on the leaf's upper side, and are abundant on the bottom. In each experiment, two treatments were considered. (1) Control—no foliar spray and (2) CO₂-enriched foliar spray. The sprayed leaves were exposed every 10 minutes and control and sprayed leaves were measured for stomatal conductance (ICT International SC-1). Treatment and measurement continued for 180 minutes. The two experiments varied in how the leaf's bottom was prepared. In the first experiment no modification was made to the leaf bottom and in the second experiment the leaf's bottom was covered with petroleum jelly to prevent stomatal conductance.

Both experiments were consistent in showing higher stomatal conductance in CO₂-exposed treatments. A T-test was used to compare stomatal conductance data. Significant differences existed between CO₂-exposed treatments in both experiments (experiment 1 (bottom untreated): p=1.369×10⁻⁹; experiment 2 (petroleum jelly): p=2.743×10⁻¹¹). See Table 6.

TABLE 6

| | Bottom Untreated | | Petroleum Jelly | |
|---|---|---|---|---|
| Min | Control | CO₂-Foliar Spray | Control | CO₂-Foliar Spray |
| 10 | 48.1 | 59 | 33.3 | 59 |
| 20 | 36.4 | 241.8 | 29.6 | 1499.8 |
| 30 | 83.3 | 913.8 | 38.7 | 2077.1 |
| 40 | 62.6 | 1351.3 | 42.7 | 1105.6 |
| 50 | 66.1 | 986.2 | 49.1 | 1115.1 |
| 60 | 130 | 1111.5 | 37.7 | 975.2 |
| 70 | 72 | 595.2 | 41.2 | 1075.9 |
| 80 | 84.9 | 1250.1 | 46.1 | 1310.9 |
| 90 | 35 | 441.6 | 50 | 1516 |
| 100 | 56.7 | 1240.3 | 84.6 | 1071.8 |
| 110 | 66.5 | 852.4 | 28.9 | 1128.4 |
| 120 | 52.6 | 730.6 | 37.8 | 932.2 |
| 130 | 36.8 | 849.6 | 47.7 | 807.5 |
| 140 | 32.8 | 649.1 | 50.3 | 1400 |
| 150 | 35.7 | 1030.4 | 35.1 | 1037.9 |
| 160 | 43.3 | 433.8 | 38.5 | 875.5 |
| 170 | 78.8 | 1120.8 | | |

Results from these experiments suggest that the CO₂ rich microenvironment surrounding the leaf created by foliar spray is capable of bypassing the leaf's cuticle. The cuticle is a waxy covering that has evolved to prevent water loss in the plant. It appears CO₂ can move through cracks in the cuticle and then cross the cells epidermal membrane through standard cellular transport processes. The enriched spray

All patent applications and publications mentioned in this document are hereby incorporated by reference herein for the teachings for which they are cited, as if fully set forth in this specification.

The invention described and claimed herein is not to be limited in scope by the specific aspects herein disclosed, since these aspects are intended as illustrations of several aspects of the invention. Any equivalent aspects are intended to be within the scope of this invention. Indeed, various modifications of the invention in addition to those shown and described herein will become apparent to those skilled in the art from the foregoing description. Such modifications are also intended to fall within the scope of the appended claims.

1. A method for increasing the growth rate of plants comprising applying a growth promoting composition to the plant as a foliar spray, wherein said growth promoting composition comprises carbon dioxide infused water.

2. The method of claim 1, wherein said growth promoting composition comprises carbon dioxide at a concentration greater than about 400 mg CO2/l water.

3. The method of claim 1, wherein said growth promoting composition comprises carbon dioxide at a concentration greater than about 1000 mg CO2/l water.

4. The method of claim 1, wherein said growth promoting composition comprises carbon dioxide at a concentration greater than about 2000 mg CO2/l water.

5. The method of claim 2 wherein said growth promoting composition is applied in a greenhouse.

6. The method of claim 2 wherein said growth promoting composition is applied outdoors.

7. The method of claim 6, wherein said growth promoting composition further comprises a spray enhancer which enhances the ability of the method to deliver CO2 to the plant.

8. The method of claim 7 wherein said spray enhancer enhances the wetting of the plant and penetration of carbon dioxide into the plant.

9. The method of claim 6, wherein said growth promoting composition is applied to plants using microporous hydrophobic hollow fibre membranes for controlling the dissolved CO2 content of an aqueous liquid containing dissolved CO2 and a foliar spray apparatus for applying said liquid to said plants.

10. The method of claim 9, wherein said foliar spray apparatus comprises an electrostatic sprayer.

11. A method according to claim 6 wherein the plant is Cannabis sativa or Cannabis indica.

12. A method according to claim 6 wherein the plant is lettuce.

13. A method according to claim 6 wherein the plant comprises microgreens.

14. A method according to claim 7 wherein the plant is grown under organic conditions.

15. A method according to claim 7 wherein the carbon dioxide infused water contains CO₂ concentrations falling within the range of about 0.37 mg/l (aq.) to about 2400 mg/l (aq.).

16. A method according to claim 7 wherein the carbon dioxide infused water contains CO₂ concentrations falling within the range of about 0.7 mg/l (aq.) to about 2000 mg/l (aq.).

17. A method according to claim 9 wherein the carbon dioxide infused water contains CO₂ concentrations greater than about 0.6 mg/liter (aq.).

18. A method according to claim 9 wherein the carbon dioxide infused water contains CO₂ concentrations greater than about 0.7 mg/liter (aq.).

19. A method according to claim 9 wherein the carbon dioxide infused water contains CO₂ concentrations greater than about 1.0 mg/liter (aq.).
